(19) United States
(12) Patent Application Publication
(10) Pub. No.: US 2021/0172965 A1
(43) Pub. Date: Jun. 10, 2021

(54) METHOD FOR MEASURING AND MANAGING RED BLOOD CELL OMEGA-3 FATTY ACID DOCOSAHEXAENOIC ACID LEVEL IN HUMAN FEMALES TO SUPPORT A TERM PREGNANCY AND REDUCE RISK OF SPONTANEOUS PRETERM BIRTH

(71) Applicant: OMEGAQUANT ANALYTICS, LLC, Sioux Falls, SD (US)

(72) Inventors: William Stephen HARRIS, Sioux Falls, SD (US); Kristina Harris Jackson, Sioux Falls, SD (US)

(73) Assignee: OMEGAQUANT ANALYTICS, LLC, Sioux Falls, SD (US)

(21) Appl. No.: 16/705,925

(22) Filed: Dec. 6, 2019

(51) Int. Cl. G01N 33/80; A61K 31/202; A61K 9/00; G01N 33/92; A23L 33/12; A23L 33/00

(52) U.S. Cl. CPC G01N 33/80; A61K 31/202; A61K 9/0056; A23V 2002/00; A23L 33/12; A23L 33/40; G01N 33/92

(57) ABSTRACT

A method for measuring and managing DHA levels in a pregnant woman to substantially reduce risk of spontaneous preterm birth, including: determining the blood level of DHA in the pregnant woman at or about the end of the first trimester or beginning of the second trimester, the DHA level being a first comparator; determining level of DHA in at least one pregnant subject at least one predetermined period, where the at least one pregnant subject having achieved a term pregnancy, and where the level of DHA at the at least one predetermined period being at least a second comparator; comparing the level of DHA in the first and at least second comparator; and when the difference between the first and the at least second comparator is greater than a predetermined value, administering to the subject a therapeutically effective amount of DHA to reduce pathology relating to spontaneous preterm birth.

### FIELD

The present invention relates to a method of measuring and managing of red blood cell omega-3-fatty acid docosahexaenoic acid (DHA) level in pregnant women in an effort to help support a term pregnancy by substantially reducing risk for spontaneous preterm birth. In particular the invention relates to diagnostic testing of blood levels of DHA during pregnancy and supplementary treatment based on outcomes of testing for a target DHA blood level, timing of testing, and to remediate sub-clinical DHA blood level during pregnancy.

The invention has been developed primarily for use in/with a pregnant human female to substantially reduce risk of a spontaneous preterm birth and will be described hereinafter with reference to this application. However, it will be appreciated that the invention is not limited to this particular field of use.

### BACKGROUND

In the United States, it is estimated that about 70% of women of childbearing age are omega-3 fatty acid deficient. Omega-3 fatty acids are natural polyunsaturated fats found in sea foods like fish and which are presently also available as dietary supplements. They contain more than one double bond in the aliphatic chain. They are named according to the number (>1), position and configuration of double bounds. The three major types of omega-3 fatty acids are alpha-linolenic acid (ALA), eicosapentaenoic acid (EPA), and docosahexaenoic acid (DHA).

It is considered important for a fetus to receive DHA in the womb as this is often associated with brain development. Large amounts of fat and DHA are transferred from the mother to the growing fetus via the placenta in the last trimester of pregnancy. Infants who are born preterm may therefore miss the placental supply of DHA and such deficiency can have detrimental effects. Preterm birth, generally understood as birth prior to 37 weeks gestation, is also a primary trigger of neonatal and infant mortality and often surviving children may suffer cognitive deficiencies and be at increased risk cardio and pulmonary diseases in adult life.

The effect of fish intake during pregnancy on the baby's cognitive function has been the focus of research for decades. A recent systematic analysis of 44 research studies including 102,944 mother-offspring pairs determined that there was “moderate and consistent evidence” that consuming seafood during pregnancy is beneficial for the baby's neurocognitive development as compared to avoiding seafood during pregnancy. It is also understood that confounding factors beyond the mother's diet during pregnancy also influence child development.

There have been suggestions of other benefits of DHA including a role in the immune response to inflammation and infection. In addition, some evidence of use of DHA-rich fish oil by pregnant women during pregnancy suggests a positive effect on the term of pregnancy and concomitant decrease rate of number of infants born preterm. However, due to non-compliance, lack of guidance from the medical community, or an inability for the patient to adhere to lifestyle modifications, optimal patient care is not achieved through current standard of care practices and other therapeutic interventions or strategies must be considered.

One object of the present invention is to manage and control DHA levels in pregnant women in an attempt to help prevent preterm birth, i.e., consistently decrease the number of children born preterm and therefore expose an unborn child to optimal brain development.

It is to be understood that, if any prior art information is referred to herein, such reference does not constitute an admission that the information forms part of the common general knowledge in the art, in Australia or any other country.

### SUMMARY

In a first aspect of the invention there is described a method for measuring and managing DHA levels in a pregnant woman to substantially reduce risk of spontaneous preterm birth, the method comprising: (a) determining the blood level of DHA in the pregnant woman at or about the end of the first trimester or beginning of the second trimester (10-20 weeks gestation), the DHA level being a first comparator; (b) determining level of DHA in at least one pregnant subject at least one predetermined period, wherein the at least one pregnant subject having achieved a term pregnancy, and wherein the level of DHA at the at least one predetermined period being at least a second comparator; (c) comparing the level of DHA in the first and at least second comparator; (d) wherein when the difference between the first and the at least second comparator is greater than a predetermined value, administering to the pregnant female subject a therapeutically effective amount of DHA to substantially reduce pathology relating to spontaneous preterm birth and its adverse effects.

Benefit of this methodology is that prenatal DHA levels are treated and maintained at early stages of pregnancy to ensure proper DHA levels substantially throughout pregnancy in an attempt to help prevent spontaneous preterm birth.

Preferably the first comparator is repeated over the term of the pregnancy at regular spaced intervals and compared with the at least second comparator to assess and regulate the levels of DHA and determine treatment regimen to elevate and maintain DHA levels.

The invention helps to aid in elongating the gestational period in a human female subject who has been found to be deficient in blood omega-3 fatty acids, specifically docosahexaenoic acid (DHA), which thereby also reduces risk for spontaneous preterm birth.

Preferably the process of testing blood levels of DHA and adapting intake based on the results can be done any time before and during pregnancy as well as postpartum for the other benefits that DHA provides for the mother and infant. Preferably the DHA analysis of blood uses a dried blood spot (DBS) to provide a (DBS) DHA level to assess the red blood cell (RBC) DHA level, wherein the dried blood spot (DBS) analysis has an improved viable term for assessment than a direct red blood cell (RBC) DHA. The improved viable term for assessment using the dried blood spot (DBS) analysis can be improved over a direct red blood cell (RBC) DHA by:

a. Extended available assessment time;

b. Reduced temperature degradation;

c. Lessening of temperature control.

In a related aspect of the invention there is described a method for managing and controlling DHA levels in a pregnant subject comprising: a. collecting a blood sample of a patient at predetermined intervals during pregnancy; b. performing a bioassay of the blood sample to determine the level of DHA in red blood cells (RBC); c. where a DHA deficiency is determined, administering an amount of DHA over a predetermined period to achieve a predetermined DHA level in RBC to substantially prevent preterm birth of an infant.

The present inventors have discovered that risk factors for preterm birth and therefore immature brain development occurs at a predetermined DHA level in RBC. Where a DHA level deficiency is identified by the diagnostic assay, remedial treatment of the subject with supplemental DHA is required to manage DHA levels to within a predetermined target range within an effective period of the predetermined DHA level in RBC, to control risk factors of preterm birth.

The diagnostic test is subsequently used to monitor the success of the supplementary administration of DHA and DHA blood level management.

In a related aspect of the present invention there is disclosed a method of treating a pregnant woman using prenatal docosahexaenoic acid (DHA) levels, the method comprising the steps of: a. collecting a blood sample from a pregnant woman; b. analysing the blood sample for a red blood cell (RBC) DHA level; c. comparing the analysed RBC DHA level to a predetermined acceptable DHA level at that period in the term of the pregnancy; wherein an analysed red blood cell (RBC) DHA level less than about 5% of total RBC fatty acids represents a deficiency; and wherein the deficiency is treated with supplemental DHA and DHA levels monitored for return to acceptable level for mitigating risk for preterm birth.

The method can further include the step of d. wherein the DHA levels in a pregnant woman are adapted to aid lowering of the risk of pre-term birth. In addition, this can continue postpartum including from supply in human milk.

The method can further include the step of: e. wherein the DHA levels in a pregnant woman are monitored by undertaking at least steps a., b., and c. at one or more different stages in the pregnancy term and the prenatal docosahexaenoic acid (DHA) levels are adapted to aid lowering of the risk of pre-term birth.

In the method of the invention, the step of collecting of the blood sample can use a dried blood spot (DBS) to provide a DBS DHA level to assess the red blood cell (RBC) DHA level wherein the dried blood spot (DBS) analysis has an improved viable term for assessment than a direct red blood cell (RBC) DHA and wherein the DBS analysis has been shown to provide an accurate estimate of the RBC DHA level.

The DBS analysis provides some advantages over direct RBC DHA testing including: a. Extended available assessment time; b. Reduced temperature degradation; c. Lessening of temperature control.

Preferably the step of analysing the blood sample for a RBC DHA level uses a predetermined ratio of the (DBS) DHA level to provide the RBC DHA level.

Preferably the step of analysing the blood sample for a red blood cell (RBC) DHA level can use as assessment of dried blood spot (DBS)DHA by a comparison of determined relative quantity of DHA fatty acid to the other 24 fatty acids wherein the sum of the fatty acids constitutes the total fatty acid content of the blood, and each individual fatty acid was expressed as a percent of the total.

Preferably the other 24 fatty acids are identified (by class): saturated (14:0, 16:0, 18:0, 20:0, 22:0 24:0); cis monounsaturated (16:1, 18:1, 20:1, 24:1); cis n-6 polyunsaturated (18:2, 18:3, 20:2, 20:3, 20:4, 22:4, 22:5); cis n-3 polyunsaturated (18:3, 20:5, 22:5, 22:6), and trans fatty acids (16:1n7t, 18:1t, 18:2t).

Preferably the DHA content of the dried blood spot is calculated as the area counts for DHA (22:6) divided by the total area counts for all 24 fatty acids, wherein DHA may be expressed as a percent.

Preferably the DHA percent from the dried blood spot analysis can be converted to the RBC DHA equivalent using the following equation: a ratio of the (DBS) DHA level to provide the red blood cell (RBC) DHA level according to:

Prenatal(RBC)DHA=dried blood spot(DBS)DHA×1.2844+0.01

with an error margin of +/−0.01

at a confidence level r=0.95

The inventors have found that a DHA level in red blood cells of less than about 5% in a subject by the third trimester of pregnancy indicates an increased risk for preterm birth. The inventors have found that the risk of preterm birth in women with low DHA blood levels, i.e. less than about 5%, is increased by a factor of at least 10-fold. Once DHA blood level in RDC is determined, the method of the invention can further include a step of adapting a treatment program for returning DHA blood levels of the subject to acceptable levels.

Preferably the step of adapting includes: a. addition of a determined quantity and portions of DHA enhancing foods in the diet; b. addition of a determined quantity and dosages of DHA enhancing substances; wherein the determined additions are commensurate with expected raising of the assessed DHA level of Prenatal (RBC)DHA to a level of about or greater than 5% by the third trimester of pregnancy.

Preferably the step of monitoring the DHA levels in a pregnant woman are at one stage in the pregnancy term prior to the third trimester. The step of monitoring the DHA levels in a pregnant woman can be spaced at different stages in the pregnancy term.

Preferably the spaced different stages in the pregnancy term are about two-three months apart. The spaced different stages in the pregnancy term can include within the first or second trimester (12-20 weeks) and near the beginning on the third trimester (28-30 weeks gestation). However, the process of testing blood levels of DHA and adapting intake based on the results can be done any time before and during pregnancy as well as postpartum for the other benefits that DHA provides during this period in the lifecycle.

Depending on determined DHA blood levels and timing of the determination within a gestational period, the subject can be treated with a managed program of supplemental DHA and DHA levels managed to progressively return within clinical RBC concentrations. One problem however is that simply increasing the intake of DHA may expose the subject to wide DHA concentration fluctuations, given personal differences in metabolic functions of mother and fetus, and place the subject in risk of serious complications such as excessive bleeding during pregnancy or delivery or even extended term pregnancy and need for emergency cesarean delivery.

In a further related aspect of the invention there is disclosed a method of treating a pregnant woman of a deficiency of docosahexaenoic acid (DHA) by the step of: a. providing determined addition of quantity and portions of DHA enhancing foods in the diet; and/or addition of a determined quantity and dosages of DHA enhancing substances; wherein the determined additions are commensurate with assessed prenatal docosahexaenoic acid (DHA) levels and an individual metabolic effectiveness of the additions so as to reach or extend above a level of about or greater than 5% by the third trimester of pregnancy.

The method of treating a pregnant woman can include the steps of: a. monitoring of the individual effectiveness of the additions determined by: i. the monitoring of assessed prenatal docosahexaenoic acid (DHA) levels at spaced different stages in the pregnancy term including within the first or second trimester, and ii. at about 2-3 months later and near to the start of the third trimester to review; and b. adjusting the determined addition of quantity and portions of DHA enhancing foods in the diet; and/or addition of a determined quantity and dosages of DHA enhancing substances as an aid to reach or extend above a level of about or greater than 5% by the third trimester of pregnancy; wherein a DHA level of about or greater than about 5% by the third trimester of pregnancy provides a decreased risk for preterm birth.

Some embodiments of the present disclosure include a method for testing prenatal docosahexaenoic acid (DHA) levels to ensure proper DHA levels in pregnant women in an attempt to help prevent preterm birth. The method may include collecting a blood sample from a pregnant woman; and analysing the red blood cells for DHA levels, wherein a DHA level less than about 5% may indicate an increased risk for preterm birth.

### DETAILED DESCRIPTION

In the following detailed description of the invention, numerous details, examples, and embodiments of the invention are described. However, it will be clear and apparent to one skilled in the art that the invention is not limited to the embodiments set forth and that the invention can be adapted for any of several applications.

The method of the present disclosure may be used to test prenatal DHA blood levels and may comprise the following elements. This list of possible constituent elements is intended to be exemplary only, and it is not intended that this list be used to limit the method of the present application to just these elements. Persons having ordinary skill in the art relevant to the present disclosure may understand there to be equivalent elements that may be substituted within the present disclosure without changing the essential function or operation of the method.

The various elements of the method of the present invention may be related in the following exemplary fashion. It is not intended to limit the scope or nature of the relationships between the various elements and the following examples are presented as illustrative examples only.

By way of example, some embodiments of the invention include a method for testing prenatal docosahexaenoic acid (DHA) blood levels to ensure proper DHA levels in pregnant women in an attempt to lower her risk for preterm birth, the method comprising collecting a blood sample and analysing the red blood cells for DHA levels, wherein if the DHA level is less than 5% the pregnant woman may be advised to increase her omega-3 DHA intake to raise blood levels.

More specifically, the method may comprise collecting a drop of blood from a pregnant woman on, for example, filter paper that is pre-treated with a 3-compound antioxidant cocktail and allowed to dry at room temperature for about 15 minutes. A paper punch of the dried blood spot may then be transferred to a container, such as a screw-top glass vial, and about 250 μL of a derivatizing reagent may be added to the container. In embodiments, the derivatizing reagent may comprise, for example, boron trifluoride in methanol (14%), toluene, and methanol (35:30:35 parts). The container may then be shaken and heated at about 100° C. for about 45 minutes in, for example, an aluminium bead hot bath.

After the mixture cools, hexane and distilled water (about 250 μL of each) may be added to the vial. After vortexing the vials briefly, the samples may be spun to separate layers, and an aliquot of the hexane layer that contains the fatty acid (FA) methyl esters may be transferred to another container, such as a via, for gas chromatography (GC). GC with flame ionization detection may be carried out using a gas chromatograph equipped with capillary column, such as a 100-m fused silica capillary column (0.25 mm internal diameter, 0.2 μm film thickness) using hydrogen as a carrier gas and nitrogen as the make-up gas.

Fatty acids may be identified by comparison with a standard mixture of fatty acid characteristics of RBCs, which may also be used to construct individual fatty acid calibration curves. The following 24 fatty acids may be identified (by class): saturated (14:0, 16:0, 18:0, 20:0, 22:0, 24:0); cis monounsaturated (16:1, 18:1, 20:1, 24:1); cis n-6 polyunsaturated (18:2, 18:3, 20:2, 20:3, 20:4, 22:4, 22:5); cis n-3 polyunsaturated (18:3, 20:5, 22:5, 22:6).

The DHA content of the dried blood spot may be calculated as the area counts for DHA (22:6) divided by the total area counts for all 24 fatty acids, wherein DHA may be expressed as a percent. The DHA percent from the dried blood spot analysis may be converted to the RBC DHA equivalent using the following equation:

Prenatal(RBC)DHA=dried blood DHA×1.2844+0.01(r=0.950)

Because prenatal RBC DHA levels less than 5% may indicate a higher than normal risk of preterm birth, a pregnant woman having an RBC DHA level less than about 5% may be advised to increase her DHA intake.

The above-described embodiments of the invention are presented for purposes of illustration and not of limitation. While these embodiments of the invention have been described with reference to numerous specific details, one of ordinary skill in the art will recognize that the invention can be embodied in other specific form without departing from the spirit of the invention. Thus, one of ordinary skill in the art would understand that the invention is not to be limited by the foregoing illustrative details, but rather is to be defined by the appended claim.

### Interpretation

### Embodiments

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the above description of example embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description of Specific Embodiments are hereby expressly incorporated into this Detailed Description of Specific Embodiments, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

Different Instances of Objects

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

Specific Details

In the description provided herein, numerous specific details are set forth. However, it is understood that embodiments of the invention may be practiced without these specific details. In other instances, well-known methods, structures and techniques have not been shown in detail in order not to obscure an understanding of this description.

### Terminology

In describing the preferred embodiment of the invention illustrated in the drawings, specific terminology will be resorted to for the sake of clarity. However, the invention is not intended to be limited to the specific terms so selected, and it is to be understood that each specific term includes all technical equivalents which operate in a similar manner to accomplish a similar technical purpose. Terms such as “forward”, “rearward”, “radially”, “peripherally”, “upwardly”, “downwardly”, and the like are used as words of convenience to provide reference points and are not to be construed as limiting terms.

Comprising and Including

In the claims which follow and in the preceding description of the invention, except where the context requires otherwise due to express language or necessary implication, the word “comprise” or variations such as “comprises” or “comprising” are used in an inclusive sense, i.e. to specify the presence of the stated features but not to preclude the presence or addition of further features in various embodiments of the invention.

Any one of the terms: including or which includes or that includes as used herein is also an open term that also means including at least the elements/features that follow the term, but not excluding others. Thus, including is synonymous with and means comprising.

### Scope of Invention

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

Although the invention has been described with reference to specific examples, it will be appreciated by those skilled in the art that the invention may be embodied in many other forms.

### INDUSTRIAL APPLICABILITY

It is apparent from the above, that the arrangements described are applicable to a method for treating a pregnant woman using prenatal docosahexaenoic acid (DHA).

What is claimed is:

1. A method for managing and controlling DHA levels in a pregnant subject comprising the steps of:
a. collecting a blood sample from a pregnant woman;
b. analysing the blood sample for a red blood cell (RBC) DHA level; and
c. comparing the analysed red blood cell (RBC) DHA level to a predetermined acceptable DHA level at that period in the term of the pregnancy;
wherein an analysed red blood cell (RBC) DHA level less than the predetermined acceptable DHA level indicates an increased risk for preterm birth.

2. The method according to claim 1 includes the step of:
d. wherein the DHA levels in a pregnant woman are adapted to aid lowering of the risk of pre-term birth.

3. The method according to claim 1 including the step of:
e. wherein the DHA levels in a pregnant woman are monitored by undertaking at least steps of claim a.-c. at one or more different stages in the pregnancy term and the prenatal docosahexaenoic acid (DHA) levels are adapted to aid lowering of the risk of pre-term birth.

4. The method according to claim 1 wherein the step of collecting of the blood sample uses a dried blood spot (DBS) to provide a (DBS) DHA level to assess the red blood cell (RBC) DHA level wherein the dried blood spot (DBS) analysis has an improved viable term for assessment than a direct red blood cell (RBC) DHA.

5. The method according to claim 1 wherein the improved viable term for assessment using the dried blood spot (DBS) analysis is improved over a direct red blood cell (RBC) DHA by:
a. Extended available assessment time;
b. Reduced temperature degradation;
c. Lessening of temperature control.

6. The method according to claim 1 wherein the step of analysing the blood sample for a red blood cell (RBC) DHA level uses a predetermined ratio of the (DBS) DHA level to provide the red blood cell (RBC) DHA level.

7. The method according to claim 6 wherein the step of analysing the blood sample for a red blood cell (RBC) DHA level uses an assessment of dried blood spot (DBS)DHA by a comparison of determined relative quantity of DHA fatty acid to the other 21 fatty acids wherein the sum of the fatty acids constitutes the total fatty acid content of the blood, and each individual fatty acid was expressed as a percent of the total.

8. The method according to claim 6, wherein the other 24 fatty acids are identified (by class): saturated (14:0, 16:0, 18:0, 20:0, 22:0 24:0); cis monounsaturated (16:1, 18:1, 20:1, 24:1); cis n-6 polyunsaturated (18:2, 18:3, 20:2, 20:3, 20:4, 22:4, 22:5); cis n-3 polyunsaturated (18:3, 20:5, 22:5, 22:6).

9. The method according to claim 6, wherein the DHA content of the dried blood spot is calculated as the area counts for DHA (22:6) divided by the total area counts for all 24 fatty acids, wherein DHA is expressed as a percent.

10. The method according to claim 7 wherein the DHA percent from the dried blood spot analysis can be converted to the RBC DHA equivalent using the following equation:
a ratio of the (DBS) DHA level to provide the red blood cell (RBC) DHA level according to:
Prenatal(RBC)DHA=dried blood spot(DBS)DHA×1.2844+0.01
with an error margin of +/−0.01
at a confidence level r=0.95

11. The method according to claim 10 wherein a DHA level of less than about 5% by the third trimester of pregnancy indicates an increased risk for preterm birth.

12. The method according to claim 11 wherein the step of adapting includes:
a. addition of a determined quantity and portions of DHA enhancing foods in the diet;
b. addition of a determined quantity and dosages of DHA enhancing substances;
wherein the determined additions are commensurate with expected raising of the assessed DHA level of Prenatal (RBC)DHA to a level of about or greater than 5% by the third trimester of pregnancy.

13. The method according to claim 3 wherein the step of monitoring the DHA levels in a pregnant woman are conducted at:
a. one stage in the pregnancy term prior to the third trimester; or
b. spaced different stages in the pregnancy term including:
i. about 2-3 months apart;
ii. within the end of the first or the start of the second trimester and at the start of the third trimester
c. prior to becoming pregnant for the purpose of optimizing DHA levels for a wanted future pregnancy

14. A method of treating a pregnant woman of a deficiency of docosahexaenoic acid (DHA) by the step of:
a. providing determined addition of quantity and portions of DHA enhancing foods in the diet; and/or addition of a determined quantity and dosages of DHA enhancing substances;
wherein the determined additions are commensurate with assessed prenatal docosahexaenoic acid (DHA) levels and an individual metabolic effectiveness of the additions so as to reach or extend above a level of about or greater than 5% by the third trimester of pregnancy.

15. The method of treating a pregnant woman according to claim 14 including the step of:
a. monitoring of the individual effectiveness of the additions determined by
i. the monitoring of assessed prenatal docosahexaenoic acid (DHA) levels at spaced different stages in the pregnancy term including within the first or second trimester and
ii. at the start of the third trimester to review, and
b. adjusting the determined addition of quantity and portions of DHA enhancing foods in the diet; and/or addition of a determined quantity and dosages of DHA enhancing substances as an aid to reach or extend above a level of about or greater than 5% by the third trimester of pregnancy
wherein a DHA level of about or greater than about 5% by the third trimester of pregnancy provides a decreased risk for preterm birth.

16. A method for measuring and managing DHA levels in a pregnant woman to substantially reduce risk of spontaneous preterm birth, the method comprising:
a. determining the blood level of DHA in the pregnant woman at or about the end of the first trimester or beginning of the second trimester (10-20 weeks gestation), the DHA level being a first comparator;
b. determining level of DHA in at least one pregnant subject at least one predetermined period, wherein the at least one pregnant subject having achieved a term pregnancy, and wherein the level of DHA at the at least one predetermined period being at least a second comparator;
c. comparing the level of DHA in the first and at least second comparator;
d. wherein when the difference between the first and the at least second comparator is greater than a predetermined value, administering to the pregnant female subject a therapeutically effective amount of DHA to substantially reduce pathology relating to spontaneous preterm birth and its adverse effects.

17. The method of claim 16 wherein the first comparator is repeated over the term of the pregnancy at regular spaced intervals and compared with the at least second comparator to assess and regulate the levels of DHA and determine treatment regimen to elevate and maintain DHA levels.

18. The method of claim 16 wherein the process of testing blood levels of DHA and adapting intake based on the results is done any time before and during pregnancy as well as postpartum for the other benefits that DHA provides for the mother and infant.

19. The method of claim 16 wherein the DHA analysis of blood uses a dried blood spot (DBS) to provide a (DBS) DHA level to assess the red blood cell (RBC) DHA level, wherein the dried blood spot (DBS) analysis has an improved viable term for assessment than a direct red blood cell (RBC) DHA.

20. The method according to claim 19 wherein the improved viable term for assessment using the dried blood spot (DBS) analysis is improved over a direct red blood cell (RBC) DHA by:
a. Extended available assessment time;
b. Reduced temperature degradation;
c. Lessening of temperature control.
